(19) United States
(12) Patent Application Publication
(10) Pub. No.: US 2011/0135768 A1
(43) Pub. Date: Jun. 9, 2011

(54) DIHYDRO-ISOALPHA ACID AND ACACIA NILOTICA EXTRACT BASED COMPOSITIONS AND METHODS FOR WEIGHT MANAGEMENT

(75) Inventors: Gary Darland, Port Orchard, WA (US); Matthew L. Tripp, Gig Harbor, WA (US); Veera Konda, Gig Harbor, WA (US)

(73) Assignee: Metaproteomics, LLC, San Clemente, CA (US)

(21) Appl. No.: 12/856,067

(22) Filed: Aug. 13, 2010

Related U.S. Application Data
(60) Provisional application No. 61/234,081, filed on Aug. 14, 2009.

(51) Int. Cl. A61K 36/48; A61P 3/04; A61P 3/00

(52) U.S. Cl. 424/757

(57) ABSTRACT

Compositions and methods to promote or maintain weight loss utilizing dihydro-isoalpha acid compounds are disclosed.

### CROSS-REFERENCE TO RELATED APPLICATIONS

This patent application claims priority to U.S. Provisional Application Ser. No. 61/234,081 filed on Aug. 14, 2009.

### BACKGROUND OF THE INVENTION

1. Field of the Invention

The present invention relates generally to compositions and methods comprising dihydro-isoalpha acid compounds and an alcoholic extract of Acacia nilotica that can be used to promote weight loss or retard weight gain in a mammal in need.

2. Description of the Related Art

Obesity and overweight may be defined as excessive or abnormal fat accumulation which may impair health. The distinction between obesity and overweight are simply matters of degree. The World Health Organization (WHO) defines “overweight” as a BMI (body mass index) equal to or greater than 25 and “obesity” as a BMI equal to or greater than 30. WHO further notes that there is evidence that the risk of chronic diseases in populations increases progressively from a BMI of 21.

WHO's 2005 projections estimated that approximately 1.6 billion adults (age 15+) were overweight; and at least 400 million adults were obese. The Organization further projects that by 2015, approximately 2.3 billion adults will be overweight and more than 700 million will be obese.

Obesity and overweight results from an energy imbalance between calories consumed and calories expended and may be attributed, in part, to a trend towards decreased physical activity (due largely to the increasingly sedentary nature of many forms of work as well as technology in the home, and more passive leisure pursuits), changing modes of transportation, and increasing urbanization coupled with a shift in diet towards increased intake of energy-dense foods that are high in fat and sugars but low in vitamins, minerals and other micronutrients.

The health consequences of such fat accumulation include increased risk of cardiovascular disease, some cancers (breast, colon, and endometrial), musculoskeletal disorders (e.g., osteoarthritis) and diabetes. (www.who.int/mediacentre/factsheets/fs311/en/index.html).

Recognition of these health consequences has steadily increased and weight management has progressed from the realm of physical aesthetics to a medical problem requiring intervention (e.g., pharmaceutical or surgical) to lifestyle management including changes in diet and exercise for the afflicted individual. One recurring problem most often associated with lifestyle management is the failure to either lose the weight initially or to maintain the weight loss over time, especially as the individual transitions to a self policed lifestyle management program.

Numerous phytochemical or herbal based weight reduction products or systems have been investigated over the years including, for example, those centered around jojoba (U.S. Pat. No. 7,138,134), Cissus, Vernonia, and Brillantasia (U.S. Pat. Nos. 7,175,589 and 7,736,675), the phytochemical Diindolylmethane (DIM), as well as its precursor, Indole-3-carbinol (I3C), and cogener, 2-(Indol-3-ylmethyl)-3,3′ diindolylmethane (LTR-1) (U.S. Pat. No. 6,534,085), or Ferula hermonis (U.S. Pat. No. 6,780,440). All have achieved a limited degree of commercial success attributable, in part, to difficulties in maintaining product use or system compliance over time. Additionally, weight control problems may occur over time in the transition from the “diet” regimen to the day-to-day “normal” lifestyle once weight has been lost.

Another area for obesity control research has centered on GLP-1 (glucagon-like peptide-1). Enteroendocrine cells have important roles in regulating energy intake and glucose homeostasis through their actions on peripheral target organs, including the endocrine pancreas. Proglucagon synthesized by L cells found in the distal ileum and colon, is posttranslationally processed into GLP-1 (glucagon-like peptide-1), a potent insulin secretagogue (Drucker D J 1998 Glucagon-like peptides. Diabetes 47:159-169). Glucagon-like peptide 1 (GLP-1)1 is known as an insulinotropic hormone exerts its effects on glucose homeostasis in regulating 1) islet hormone function (insulin and glucagon), 2) nutrient delivery, and 3) food intake. Intra-cerebroventricular administration of GLP-1 was found to inhibit eating and reduce body weight in rats (Turton M. D, 1996; Meeran K, 1999).

In human clinical trials, liraglutide, a stable GLP-1 mimic (2 amino acid changes in GLP-1, 97% homology) dose dependently reduced the body weight with cardiovascular beneficial effects such as reduced blood pressure and triglyceride content in patients with T2DM (reviewed in Pratley 2008, Sulistio, 2009).

GLP-1 levels increase in blood within minutes of food intake, well before any food appears in the gut, suggesting a neural signal to the L cells (Drucker 2006). GLP-1, a 30 amino acid peptide derived from proglucagon gene. GLP-1 was shown to regulate by multiple mechanism including proglucagon gene regulation, DPP-4 inhibition and G protein-coupled receptor (GPR) signaling pathways. Many factors including glucose load, oils (corn and perilla), and non-digestable carbohydrates involved in GLP-1 secretion (Irini 1999, Cani, 2007, Lu 2008).

Recently, it was reported that unsaturated long-chain fatty acids (such as a-linolenic acid) promote the secretion of GLP-1 via GPR120, which is expressed predominantly in the colon (Hirasawa A, 2008). M2 receptors antagonist (atropine & Pirenzepine) block the corn oil induced GLP-1 suggesting the role of M2 receptors in GLP-1 activation (Anini 2002). It was reported that bitter taste receptors T1R (Dyer, 2005) and T2R (Wu, 2002, Jeon, 2008) express in the gut and stimulate the release of hormones. Also, GPR 119 involved in glycemic control by enhancing GLP-1 in intestinal endocrine cells (Chu 2008) and shown beneficial in treatment of T2D and obesity (Overton, 2008). These data suggest that multiple GPR regulate the GLP-1 secretion in L cells.

In addition to potentiating glucose-induced insulin secretion, GLP-1 stimulates proinsulin gene expression and biosynthesis (Drucker D J, Philippe J, Mojsov S, Chick W L, Habener J F 1987). Glucagon-like peptide I stimulates insulin gene expression and increases cyclic AMP levels in a rat islet cell line. Proc Natl Acad Sci USA 84:3434-3438). Furthermore, GLP-1 has been recently shown to promote satiety and reduce food intake through interactions with the hypothalamus (Turton M D, O'Shea D, Gunn I, et al. 1996, Nature 379:69-72; Flint A, Raben A, Astrup A, Holst J J, 1998 J Clin Invest 101:515-520).

The inventors had discovered methods and compositions comprising substituted 1,3-cyclopentadione compounds that can be used to promote weight loss directly or modulate weight gain in animals.

### SUMMARY OF THE INVENTION

The present invention relates generally to compositions and methods comprising dihydro-isoalpha acid compounds and an alcoholic extract of Acacia nilotica that can be used to promote weight loss or retard weight gain in a mammal in need.

A first embodiment of the invention describes methods to promote healthy weight management in a mammal in need thereof, said method comprising administering a composition comprising a therapeutically effective amount of a dihydro-isoalpha acid and an alcoholic extract of Acacia nilotica.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 graphically depicts the weight gain corrected for baseline of mice treated with Meta352 and maintained on high fat diet. Control mice, C57BL6/J (n=12/group), were maintained on a standard chow diet (LFD) or high-fat diets (HFD) in which rosiglitazone (0.5 mg kg⁻¹ d⁻¹) or Meta352 (100 mg kg-1 d-1) were incorporated. The animals were weighed weekly. At the termination of the experiment the liver, epididymal and subcutaneous adipose tissue (eAT and scAT) were dissected and weighed.

FIG. 2 graphically depicts the changes in liver weight in C57BL6/J mice maintained on a standard chow diet (LFD) or high-fat diets (HFD) alone or in which rosiglitazone (0.5 mg kg⁻¹ d⁻¹), THIAA (100 mg kg⁻¹ d⁻¹), or Insinase (100 mg kg⁻¹ d⁻¹) were incorporated or switched to alternative diets as indicated. * denote statistically significant changes in body weight relative to HFD diet alone; →denotes switching or changing diets.

FIG. 3 graphically depicts the changes in gonadal fat weight in C57BL6/J mice maintained on a standard chow diet (LFD) or high-fat diets (HFD) alone or in which rosiglitazone (0.5 mg kg⁻¹ d⁻¹), THIAA (100 mg kg⁻¹ d⁻¹), or Insinase (100 mg kg⁻¹ d⁻¹) were incorporated or switched to alternative diets as indicated. * denote statistically significant changes in body weight relative to HFD diet alone; →denotes switching or changing diets.

FIG. 4 graphically depicts the changes in subcutaneous fat weight in C57BL6/J mice maintained on a standard chow diet (LFD) or high-fat diets (HFD) alone or in which rosiglitazone (0.5 mg kg⁻¹ d⁻¹), THIAA (100 mg kg⁻¹ d⁻¹), or Insinase (100 mg kg⁻¹ d⁻¹) were incorporated or switched to alternative diets as indicated. * denote statistically significant changes in body weight relative to HFD diet alone; →denotes switching or changing diets.

FIG. 5 graphically depicts representative mice after twelve weeks on a standard chow diet (LFD) or high-fat diets (HFD) alone or in which rosiglitazone (“Rosi”; 0.5 mg kg⁻¹ d¹), THIAA (100 mg kg⁻¹ d⁻¹), or Insinase (100 mg kg⁻¹ d⁻¹) were incorporated.

### DETAILED DESCRIPTION OF THE INVENTION

The present invention relates generally to compositions and methods comprising dihydro-isoalpha acid compounds and an alcoholic extract of Acacia nilotica that can be used to promote weight loss or retard weight gain in a mammal in need.

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of recombinant DNA technology include Sambrook et al., Molecular Cloning: A Laboratory Manual, 2nd Ed., Cold Spring Harbor Laboratory Press, New York (1989); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 11th Ed., McGraw Hill Companies Inc., New York (2006).

In the specification and the appended claims, the singular forms include plural referents unless the context clearly dictates otherwise. As used in this specification, the singular forms “a,” “an” and “the” specifically also encompass the plural forms of the terms to which they refer, unless the content clearly dictates otherwise. Additionally, as used herein, unless specifically indicated otherwise, the word “or” is used in the “inclusive” sense of “and/or” and not the “exclusive” sense of “either/or.” The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable that is inherently discrete, the variable can be equal to any integer value of the numerical range, including the end-points of the range. Similarly, for a variable that is inherently continuous, the variable can be equal to any real value of the numerical range, including the end-points of the range. As an example, a variable that is described as having values between 0 and 2, can be 0, 1 or 2 for variables that are inherently discrete, and can be 0.0, 0.1, 0.01, 0.001, or any other real value for variables that are inherently continuous.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instances, well known process operations have not been described in detail, in order not to unnecessarily obscure the present invention.

Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

A first embodiment of the invention describes methods to promote healthy weight management in a mammal in need thereof, said method comprising administering a composition comprising a therapeutically effective amount of a dihydro-isoalpha acid and an alcoholic extract of Acacia nilotica.

In some aspects of this embodiment the dihydro-isoalpha acid is selected from the group consisting of dihydro-isohumulone, dihydro-isocohumulone, and dihydro-isoadhumulone. In additional aspects the composition comprises from 50 mg to 10,000 mg of the dihydro-isoalpha acid. In yet other aspects the composition further comprises from 50 mg to 10,000 mg of the alcoholic extract of Acacia nilotica.

In other aspects of this embodiment, the method promotes healthy weight management by promoting weight loss or retarding weight gain in a mammal in need. In some aspects, the mammal in need has a condition selected from the group consisting of diabetes, cardiovascular disease, dyslipidemia, hypertension, erectile dysfunction, polycystic ovary syndrome, end stage renal disease, osteoporosis, nonalcoholic steatohepatitis, obesity, and sleep apnea.

As used herein, “promoting health weight management” or variations thereof refers to usages wherein weight loss is encouraged (“promoting weight loss”) or retarding weight gain. Contemplated usage to retard weight gain include prophylactic use wherein the invention is used prior to ingestion of a high fat/high caloric meal to stave off weight gain from that meal, or use in a maintenance mode subsequent to weight loss from any diet regimen or treatment as the individual transitions from the active weight loss program and resumes a more “normal” (at least for that individual) diet regimen.

Contemplated use of the invention includes usage, without limitation, wherein individual weight loss is a direct goal (e.g., obesity); where weight loss reduces body stress (e.g., osteoporosis) or a side effect from another treatment modality (e.g., diabetes). In other instances, the weight loss may be associated with improvement of risk factors associated with a disease or condition (e.g., erectile dysfunction).

The term “pharmaceutically acceptable” is used in the sense of being compatible with the other ingredients of the compositions and not deleterious to the recipient thereof.

As used herein, “Insinase” or “Meta352” shall refer to a composition comprising dihydro-isoalpha acids and an alcoholic extract of Acacia nilotica.

As used herein, dihydro-isoalpha acids refers to those compounds generally described as reduced isoalpha acids commonly associated with hops and beer production, more specifically dihydro-isoalpha acids refers to compounds of Genus A

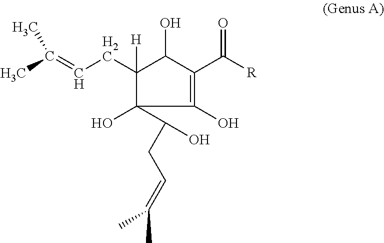

wherein dihydro-isohumulone (R=—CH₂CH(CH₃)₂); dihydro-isocohumulone (R=—CH(CH₃)₂); and dihydro-isoadhumulone (R=—CH(CH₃)CH₂CH₃) are shown. “Rho” refers to those reduced isoalpha acids wherein the reduction is a reduction of the carbonyl group in the 4-methyl-3-pentenoyl side chain.

As used herein, “RIAA” refers to any mixture of one or more of dihydro-isoadhumulone, dihydro-isocohumulone and dihydro-isohumulone.

As used herein, “compounds” may be identified either by their chemical structure, chemical name, or common name. When the chemical structure and chemical or common name conflict, the chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated or identified compounds. The compounds described also encompass isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated or N-oxides. Certain compounds may exist in multiple crystalline or amorphous forms. Also contemplated within the scope of the invention are congeners, analogs, hydrolysis products, metabolites and precursor or prodrugs of the compound. In general, unless otherwise indicated, all physical forms are equivalent for the uses contemplated herein and are intended to be within the scope of the present invention.

Compounds according to the invention may be present as salts. In particular, pharmaceutically acceptable salts of the compounds are contemplated. A “pharmaceutically acceptable salt” of the invention is a combination of a compound of the invention and either an acid or a base that forms a salt (such as, for example, the magnesium salt, denoted herein as “Mg” or “Mag”) with the compound and is tolerated by a subject under therapeutic conditions. In general, a pharmaceutically acceptable salt of a compound of the invention will have a therapeutic index (the ratio of the lowest toxic dose to the lowest therapeutically effective dose) of 1 or greater. The person skilled in the art will recognize that the lowest therapeutically effective dose will vary from subject to subject and from indication to indication, and will thus adjust accordingly.

The compounds according to the invention are optionally formulated in a pharmaceutically acceptable vehicle with any of the well known pharmaceutically acceptable carriers, including diluents and excipients [see Remington's Pharmaceutical Sciences, 18th Ed., Gennaro, Mack Publishing Co., Easton, Pa. 1990 and Remington: The Science and Practice of Pharmacy, Lippincott, Williams & Wilkins, 1995]. While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compound of the invention), as well as any other pharmacologically active ingredient useful for the treatment of the symptom/condition being treated.

The compounds of the present invention may be provided in a pharmaceutically acceptable vehicle using formulation methods known to those of ordinary skill in the art. The compositions of the invention can be administered by standard routes, though preferably administration is by inhalation routes. The compositions of the invention include those suitable for oral, inhalation, rectal, ophthalmic (including intravitreal or intracameral), nasal, topical (including buccal and sublingual), vaginal, or parenteral (including subcutaneous, intramuscular, intravenous, intradermal, and intratracheal). In addition, polymers may be added according to standard methodologies in the art for sustained release of a given compound.

Formulations suitable for administration by inhalation include formulations that can be dispensed by inhalation devices known to those in the art. Such formulations may include carriers such as powders and aerosols. The present invention encompasses liquid and powdered compositions suitable for nebulization and intrabronchial use, or aerosol compositions administered via an aerosol unit dispensing metered doses (“MDI”). The active ingredient may be formulated in an aqueous pharmaceutically acceptable inhalant vehicle, such as, for example, isotonic saline or bacteriostatic water and other types of vehicles that are well known in the art. The solutions are administered by means of a pump or squeeze-actuated nebulized spray dispenser, or by any other conventional means for causing or enabling the requisite dosage amount of the liquid composition to be inhaled into the patient's lungs. Powder compositions containing the anti-inflammatory compounds of the present invention include, by way of illustration, pharmaceutically acceptable powdered preparations of the active ingredient thoroughly intermixed with lactose or other inert powders acceptable for intrabronchial administration. The powder compositions can be administered via a dispenser, including, but not limited to, an aerosol dispenser or encased in a breakable capsule which may be inserted by the patient into a device that punctures the capsule and blows the powder out in a steady stream. Aerosol formulations for use in the subject method typically include propellants, surfactants, and co-solvents and may be filled into conventional aerosol containers that are closed by a suitable metering valve.

Formulations of compositions of the present invention suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, for example via a nasal spray, aerosol, or as nasal drops, include aqueous or oily solutions of the compound of the invention.

For oral administration, the compositions of the invention may be presented as discrete units such as capsules, caplets, gelcaps, cachets, pills, or tablets each containing a predetermined amount of the active ingredient as a powder or granules; as a solution or a suspension in an aqueous liquid or a non-aqueous liquid; or as an oil-in-water liquid emulsion or a water-in-oil emulsion and as a bolus, etc. Alternately, administration of a composition of all of the aspects of the present invention may be effected by liquid solutions, suspensions or elixirs, powders, lozenges, micronized particles and osmotic delivery systems.

Formulations of compositions according to the aspects of the present invention suitable for parenteral administration include aqueous and non-aqueous sterile injection solutions which may contain antioxidants, stabilizers, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface active or dispersing agent. Molded tablets may be made by molding, in a suitable machine, a mixture of the powdered compound moistened with an inert liquid diluent. The tablets may be optionally coated or scored and may be formulated to provide a slow or controlled release of the active ingredient therein.

Formulations of compositions of the present invention for rectal administration may be prepared as a suppository with a suitable base comprising, such as, for example, cocoa butter.

Formulations of compositions of the present invention suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier. Formulations of compositions of the present invention suitable for topical administration to the skin may be presented as ointments, creams, gels, lotions and pastes comprising the ingredient to be administered in a pharmaceutical acceptable carrier. A topical delivery system contemplated is a transdermal patch containing the ingredient to be administered.

Formulations of compositions according to the aspects of the present invention suitable for vaginal administration may be presented as pessaries, suppositories, tampons, creams, gels, pastes, foams or spray formulations containing in addition to the compound of the invention such pharmaceutically acceptable carriers as are known in the art to be appropriate.

The methods and compositions of the present invention are intended for use with any mammal that may experience the benefits of the methods of the invention. Foremost among such mammals are humans, although the invention is not intended to be so limited, and is applicable to veterinary uses. Thus, in accordance with the invention, “mammals” or “mammal in need” include humans as well as non-human mammals, particularly domesticated animals including, without limitation, cats, dogs, and horses.

As used herein, “treating” is meant reducing, preventing, and/or reversing the symptoms in the individual to which a compound of the invention has been administered, as compared to the symptoms of an individual not being treated according to the invention. A practitioner will appreciate that the compounds, compositions, and methods described herein are to be used in concomitance with continuous clinical evaluations by a skilled practitioner (physician or veterinarian) to determine subsequent therapy. Hence, following treatment the practitioners will evaluate any improvement in reducing cardiovascular risk factors or associated dyregularities according to standard methodologies. Such evaluation will aid and inform in evaluating whether to increase, reduce or continue a particular treatment dose, mode of administration, etc.

It will be understood that the subject to which a compound of the invention is administered need not suffer from a specific traumatic state. Indeed, the compounds of the invention may be administered prophylactically, prior to any development of symptoms. The term “therapeutic,” “therapeutically,” and permutations of these terms are used to encompass therapeutic, palliative as well as prophylactic uses. Hence, as used herein, by “treating or alleviating the symptoms” is meant reducing, preventing, and/or reversing the symptoms of the individual to which a compound of the invention has been administered, as compared to the symptoms of an individual receiving no such administration.

The term “therapeutically effective amount” is used to denote treatments at dosages effective to achieve the therapeutic result sought. Furthermore, one of skill will appreciate that the therapeutically effective amount of the compound of the invention may be lowered or increased by fine tuning and/or by administering more than one compound of the invention, or by administering a compound of the invention with another compound. See, for example, Meiner, C. L., “Clinical Trials: Design, Conduct, and Analysis,” Monographs in Epidemiology and Biostatistics, Vol. 8 Oxford University Press, USA (1986). The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect.

It will be appreciated by those of skill in the art that the number of administrations of the compounds according to the invention will vary from patient to patient based on the particular medical status of that patient at any given time including other clinical factors such as age, weight and condition of the mammal and the route of administration chosen.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### EXAMPLES

### Example 1

### Effects of Dietary Insinase on Weight Loss and Weigh Maintenance

The purpose of this experiment was to determine the effects of Insinase on weight loss and weight maintenance.

Animal handling and diets: C57B16/J male mice were 12 weeks old at the start of the experiment. Twelve mice were maintained 4/cage. Standard diets were purchased from Research Diets, Inc (New Brunswick, N.J.). The caloric composition of the control diet (“LFD”)(D12450B) was 20:70:10 (protein: carbohydrate: fat) and that of the high fat diet (“HFD) (D12451) was 20:35:45. The incremental increase in fat was provided by lard. After establishing that there was taste aversion, the high fat diet was supplemented with the test agents. Body weight and food consumption were determined at weekly intervals and adjustments were made if necessary to insure that the amount of active material THIAA or Insinase (100 mg kg⁻¹ d¹) and rosiglitazone (0.5 mg kg⁻¹ d⁻¹) were delivered.

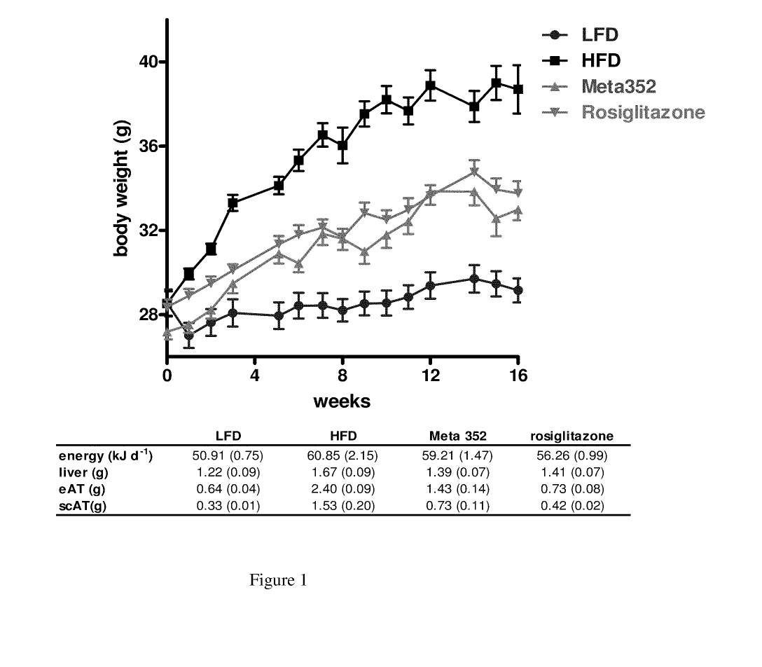

Results: At three weeks, all compounds tested resulted in significantly lowered body weight as compared to HFD diet alone. Further, by the sixteenth week, animals on an HFD+Insinase diet displayed total body weights which were significantly different than those for control mice on a LFD alone (FIG. 1).

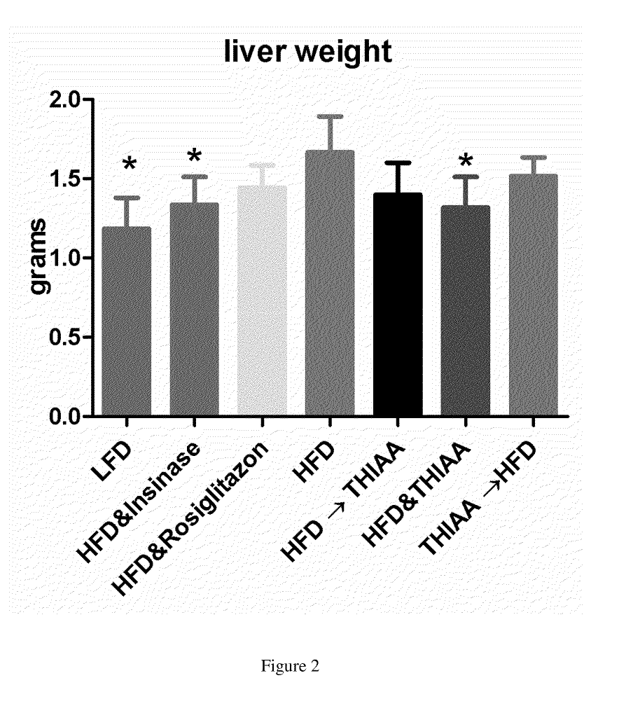

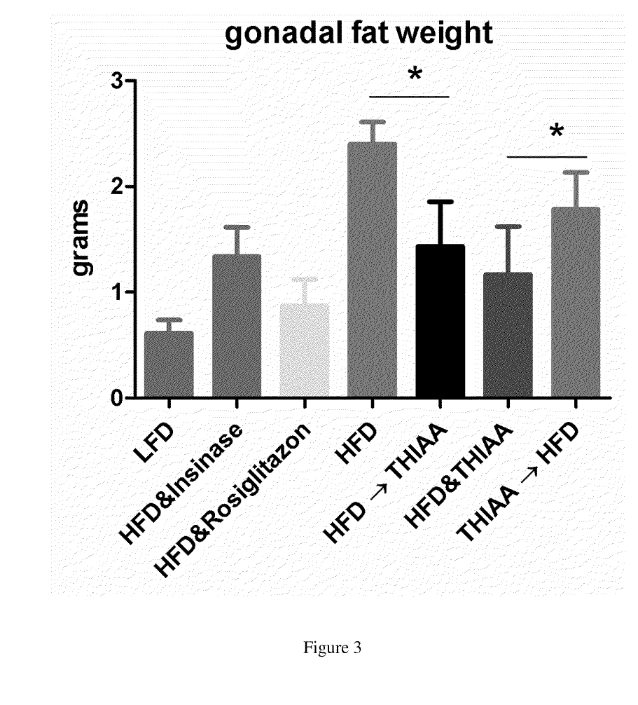

Significant reduction in liver weight (FIG. 2) was observed in the HFD+Insinase animals as compared to HFD alone as well as for gonadal fat weight (FIG. 3).

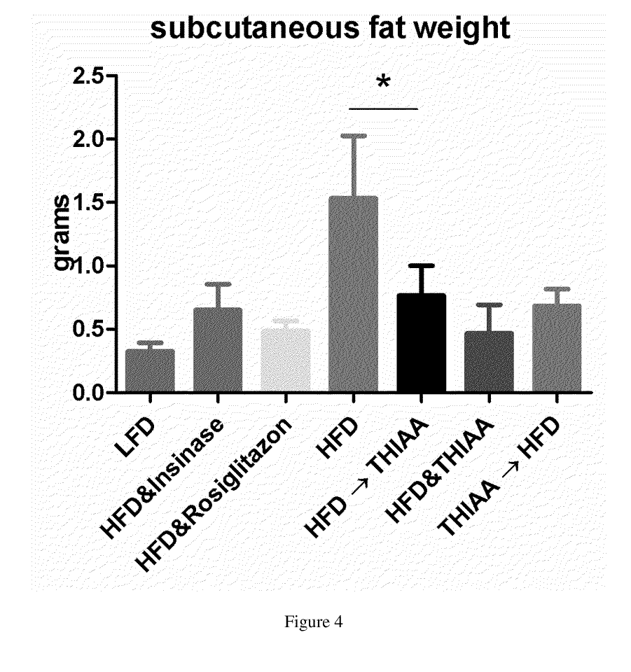

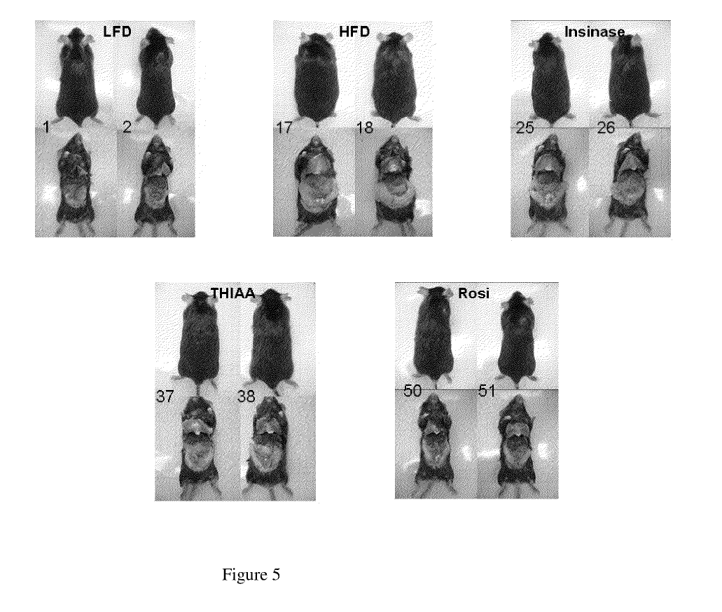

It was further noted that all compounds tested resulted in significant loss of subcutaneous body fat as compared to HFD alone (FIG. 4), which are readily apparent visually (FIG. 5).

While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims.

1. A method to promote healthy weight management in a mammal in need thereof, said method comprising administering a composition comprising a therapeutically effective amount of a dihydro-isoalpha acid and an alcoholic extract of Acacia nilotica.

2. The method according to claim 1, wherein said dihydro-isoalpha acid is selected from the group consisting of dihydro-isohumulone, dihydro-isocohumulone, and dihydro-isoadhumulone

3. The method according to claim 1, wherein the composition comprises from 50 mg to 10,000 mg of the reduced isoalpha acid.

4. The method according to claim 1, wherein the composition further comprises from 50 mg to 10,000 mg of the alcoholic extract of Acacia nilotica.

5. The method according to claim 1, wherein promoting healthy weight management is selected from promoting weight loss or retarding weight gain in a mammal in need thereof.

6. The method according to claim 1, wherein the subject in need has a condition selected from the group consisting of diabetes, cardiovascular disease, dyslipidemia, hypertension, erectile dysfunction, polycystic ovary syndrome, end stage renal disease, osteoporosis, nonalcoholic steatohepatitis, obesity, and sleep apnea.

7. The method according to claim 1, wherein said administration reduces food cravings or increases satiety.
